(19) United States
(12) Patent Application Publication
(10) Pub. No.: US 2011/0106249 A1
(43) Pub. Date: May 5, 2011

(54) SELF SUPPORTING AND FORMING BREAST IMPLANT AND METHOD FOR FORMING AND SUPPORTING AN IMPLANT IN A HUMAN BODY

(76) Inventor: Hilton Becker, Boca Raton, FL (US)

(21) Appl. No.: 12/766,821

(22) Filed: Apr. 23, 2010

Related U.S. Application Data
(63) Continuation-in-part of application No. 12/552,353, filed on Sep. 2, 2009.
Application No. 12/552,353 is a continuation of application No. 12/556,050, filed on Sep. 9, 2009.

(51) Int. Cl. A61F 2/12; B23P 11/00

(52) U.S. Cl. 623/8; 29/428

(57) ABSTRACT

A surgical implant namely a tissue expander or adjustable implant having a synthetic mesh loosely attached to the surface. The mesh is elastic in all directions and the implant adjustable or expandable. The expandable implant is filled to an over filled volume into the surrounding tissue until a new capsule forms. At this stage the volume of the implant is reduced leaving the supporting capsule with the mesh at a larger volume. The volume of the implant is then reduced thus rendering a tension free capsule around the implant. The filling tube of the adjustable implant is then removed leaving the adjustable implant in position. Alternatively the tissue expander may be removed and a new implant placed in the expanded tension free pocket.

This application is a Continuation-in-Part of my co-pending U.S. application Ser. No. 12/552,353, filed Sep. 2, 2009, and my Continuation-in-Part application Ser. No. 12/556,050 filed Sep. 9, 2009, herein incorporated by reference.

### FIELD OF THE INVENTION

This invention relates to a self forming and self supporting implant for breast reconstruction following a mastectomy, breast augmentation or the treatment of breast implant complications especially capsular contraction and more particularly to a method for forming and supporting a breast implant in a human body. The implant used is a tissue expander or an adjustable implant as disclosed in my earlier patents.

### BACKGROUND FOR THE INVENTION

Implants for breast augmentation and/or reconstruction are well known and have been in use for over 20 years. During that period, the implants have undergone a number of significant changes. For example, early implants had a smooth outer shell; however, as developments progressed the smooth shell was replaced with a textured surface. This was done in an effort to reduce problems associated with capsular contraction and to support a natural or pear shaped implant in position.

One approach to the use of a textured surface is disclosed in my co-pending U.S. patent application Ser. No. 12/169,000 filed on Jul. 8, 2008, that is a Continuation-In-Part of U.S. patent application Ser. No. 12/026,032 filed on Feb. 5, 2008. As disclosed therein, a method for texturing the surface of a synthetic implant includes the steps of providing an implant having a textured outer layer of silicone elastomer having a plurality of cavities filled with tissue growth enhancing material. Portions of the tissue growth enhancing materials protrude outwardly from the filled cavities. The implant also includes a hollow core filled with a fluid gel or liquid of silicone, saline or soy and a layer or mass of a biologically active non-absorbable material such as non-absorbable acellular dermis. The method also includes the step of forming a capsular pouch from the mass of biologically active non-absorbable material, placing the implant into the pouch and implanting the pouch containing the implant behind the breast thus holding the implant in position and reducing capsular contraction by the surrounding tissue and blood and blood vessels growing into the acellular dermis. In a preferred embodiment of my earlier invention the acellular dermis, collagen are combined with hyaluronic acid and partially impregnated in the outer layer of a silicone elastomer so that the patient's blood vessels and tissue grow into the biologically active non-absorbable or only partially absorbable filled cavities to thereby anchor the implant in place.

Essentially an implant consists of a smooth silicon bag filled with either silicon gel or saline. When inserted into the body the implant is walled off by the response of the human tissue. This is commonly referred to as encapsulation. As the capsule that is formed is scar tissue, it is fairly rigid and in certain cases may actually contract resulting in hardening around the implant. This often requires further surgery with unpredictable results. The incidence of capsular contracture is approximately 20% and is even higher in patients undergoing mastectomy requiring breast reconstruction. Besides causing tightening around the implant, contracture leads to displacement, pain, distortion and discomfort. This reaction to the implant is known as a foreign body reaction. It is commonly seen in all biological tissue, as for example an oyster forming a pearl around a grain of sand. In the human, capsular contracture may become so severe that calcification actually occurs. The implant then becomes palpable and distorted.

In an effort to decrease capsular contracture it was believed that texturing the surface of the implant would disrupt the capsular tissue, thus resulting in less capsular contracture. For example, a U.S. Pat. No. 6,913,626 advocates covering the implant with a bio absorbable material in an attempt to reduce capsular contracture. Another U.S. Pat. No. 4,648,880 utilized a woven messed draped around the implant in an attempt to reduce capsular formation.

Texturing of the outer surface of the implant has not shown much success in decreasing capsular contracture, so much so that most surgeons have now reverted back to smooth implants. However, none of the prior art attempts to disrupt the collagen fibers in the capsule, have advocated created a larger capsule which will form around the implant following which the implant can be reduced in volume which would then reduce the incidence of capsular contracture. None of the prior art suggest the use of a tissue expander combined with a mesh that would become integrated into the capsule at a larger volume and subsequently reduce the volume of the implant. This technique has been performed by the inventor on multiple surgical cases with excellent results. Thus, it has now been found that the most desirable implant is a smooth implant that results in a soft, natural feel. In summary this technique is the use of a smooth implant that is left at a smaller volume than the matured mesh supported capsule.

Notwithstanding the above it is Applicant's belief that there is a need and a potential market for an improved textured or smooth surface implant and a method for breast implant reconstruction and augmentation in accordance with the present invention. There should be a need and a market for such implants because they provide better anchoring, shape enhancement and less problems with capsular contraction. Further, it is believed that such implants can be marketed at a competitive price, are durable, improve the rate of healing and lead to more satisfactory results.

### BRIEF SUMMARY OF THE INVENTION

In essence a self supporting breast implant for breast augmentation and/or reconstruction comprises and/or consists of a generally cone shape support and an implant disposed within the support. In a preferred embodiment of the invention, the support is formed from a sheet of ULTRAPRO® partially absorbable light weight surgical mesh consisting of about 75% polypropylene (non-absorbable) and 25% poliglecaprone (absorbable) monofilament materials, available from Ethicon Inc., a Johnson and Johnson company located in Langhorne, Pa. and infinit meshPTFE available from Gore located in Flagstaf, Ariz. In practice, a disc shaped piece of mesh has a triangular piece removed and the edges left by the removal of the triangular piece are joined together to form a three dimensional cone shaped support. The implant comprises a silicone shell, a hollow core and a silicone or saline fluid dispersed in the hollow core. The implant is placed within the cone and because of the flexibility of the mesh forms a generally pear or naturally shape of a breast. The mesh also includes means such as an overlap of the mesh material for suturing to a patient's tissue or in the alternative including two or three absorbable hooks.

The invention also contemplates a method for forming and supporting a breast implant including the steps of providing a breast implant and a mass of partially absorbable light weight surgical mesh. The surgical mesh is formed into a generally cone shaped support and the implant, a smooth sided, non-absorbable shell is disposed in the cone shaped support. In a preferred embodiment of the invention the base of the cone is enclosed with a sheet of the mesh material so that the implant, preferably having a smooth surface, is fully covered by the mesh support that is in close proximity thereto and the mesh support or an extension thereof is surgically attached to the patient's tissue to thereby provide an internal bra-like support. Also, because of the flexibility of the mesh material, the cone shaped mesh in combination with the generally round or slightly pear shaped implant takes on the natural form of a breast. Ideally the mesh support pouch is used with an adjustable implant. With an adjustable gel implant, the implant can be expanded to further enhance the shape.

In a preferred embodiment of the present invention the implant comprises a tissue expander or an adjustable implant comprising an implant having an upper portion and a lower portion and wherein the implant lower portion is supported by a cup or sling shaped mesh support member supporting and surrounding a lower portion of said implant while leaving the upper portion thereof free of the support. The mesh support is adaptable to hold the implant in position with respect to a muscle underlying an incision without piercing the implant. An important aspect of this preferred embodiment resides in an expandable support that is elastic in all directions that can be expanded to enable a larger mesh supported capsule to be formed. This enables the surgeon to place a tissue expander or adjustable implant at the time of surgery, expand the implant, allow incorporation of the meshed outer layer and then reduce the volume thus resulting in a soft, pliable implant without surrounding tissue capsular contracture.

The invention will now be described in connection with the following drawings wherein like numbers have been used to identify like parts.

### DESCRIPTION OF THE DRAWINGS

FIG. 1 is a schematic illustration of a generally cone shaped support;

FIG. 2 is a schematic illustration of an implant dispersed in a mesh support in accordance with the present invention;

FIG. 3 is a flow chart illustrating a method for forming and supporting a breast implant in accordance with the present invention;

FIG. 4 is a cross-sectional view of a breast including a breast implant disposed therein in accordance with a preferred embodiment of the invention;

FIG. 5 is a schematic illustration of an implant with a mesh pouch enclosing the implant and including absorbable hooks in a rear portion thereof for attachment to a patient's tissue;

FIG. 6 is a schematic cross-sectional view of a breast with a dual lumen expandable mammary prosthesis implanted therein;

FIG. 7 is a schematic illustration of a breast implant in accordance with a preferred embodiment of the invention;

FIG. 8 is a schematic illustration of a smooth inflatable implant with a textured mesh layer of partially absorbable layer thereon;

FIG. 9 is a schematic illustration of an expanded implant and textured mesh surface wherein the volume of the implant has been expanded by adding saline or the like that allows human tissue to grow into the mesh coating;

FIG. 10 is a schematic illustration of a smooth inflatable implant with a reduced volume to provide a space between the implant and the textured mesh layer and capsular surface.

FIG. 11 is an expanded portion of the breast and implant shown in FIG. 10;

FIG. 12 is a schematic illustration of a breast implant enveloped by a mesh bag or pouch held in place with sutures;

FIG. 13 is a schematic illustration of a volume reduced implant with the mesh fixed in a tissue splinting capsule at a larger volume than the implant; and

FIG. 14 is a schematic illustration of an expanded implant forcing the mesh into human tissue.

### DESCRIPTION OF THE PREFERRED EMBODIMENTS OF THE INVENTION

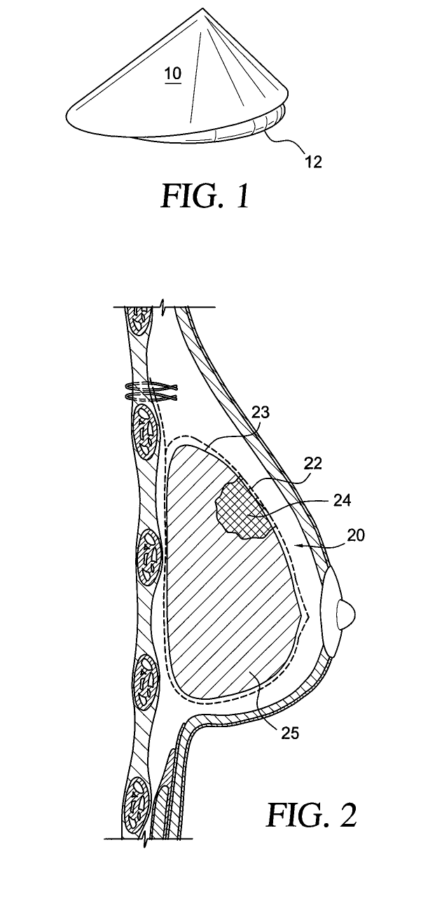

As illustrated in FIG. 1 a generally cone shaped support member 10 is formed from a mass or sheet of medical mesh as for example ULTRAPRO®, partially absorbable material from Ethicon Inc., a Johnson and Johnson company located in Langhorne, Pa. This mesh which is used in the preferred embodiment of the invention is constructed of a combination of polypropylene, about (non-absorbable) and poliglecaprone, (absorbable), monofilament material in varying percentages i.e. 80% absorbable and 20% permanent.

The absorbable portion of the mesh may encompass the whole surface, or part of the surface i.e. the upper portion can be predominantly absorbable while the lower part is predominantly non absorbable, this will allow a sling of support to remain while the upper portion is absorbed. The direction of the elasticity can also be configured so as to enhance shape of a round implant on expansion, i.e. if the anterior lower portion is made more elastic than the upper portion, so that a round implant will assume a pear shape

It is contemplated that other synthetic meshes may be used as for example polypropolene mesh with filament diameters ranging from 0.08 mm to 0.20 mm, pore size from about 0.8 to 3.0 mm, and finer, and weights from 25 to 100 grams per square meter. Other materials include polyester felt, polyester knitted mesh, polytetrafluoethylene, nylon, etc. In addition, various other types of mesh may be used in forming a support. For example, biological mesh made from collagen sheets of human and animal origin, synthetic woven mesh as for example nylon, Dacron, Gore-Tex and combination meshes with strands of nylon interwoven with strands of collagen.

The generally cone shaped support may be formed by taking a disc shaped piece of mesh and removing a triangular portion from the disc. The edges of the pie shape with a piece removed are then joined together to form a generally three dimensional cone shape as shown in FIG. 1. As shown, the cone shaped member 10 is placed on top of a silicone shell 12 that is filled with a silicone or saline fluid. In a preferred embodiment of the invention, the implant is partially enclosed in a bag or sling like support that includes the cone shaped structure.

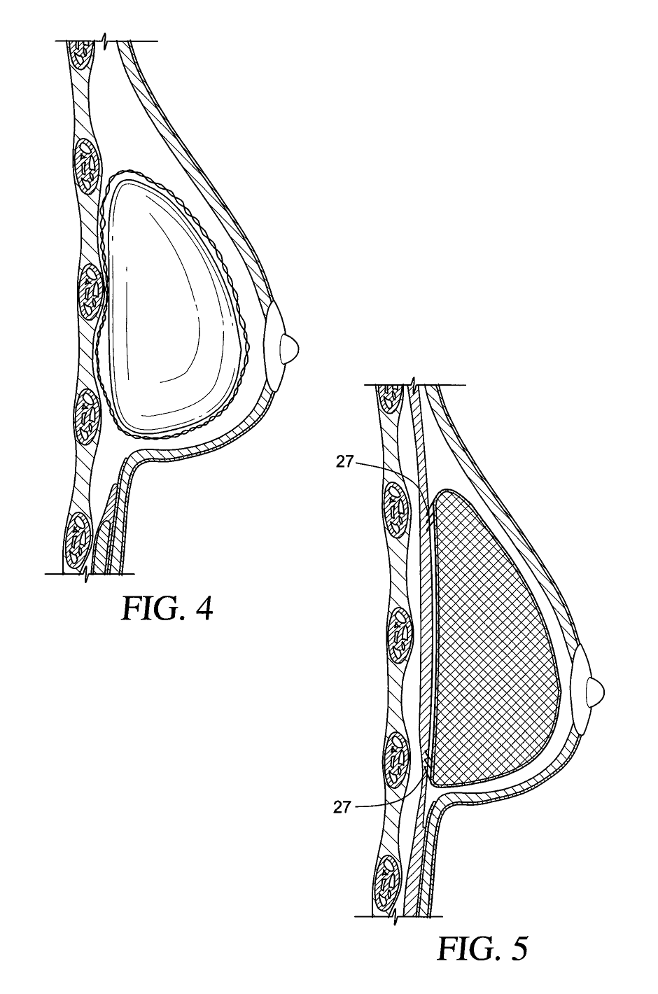

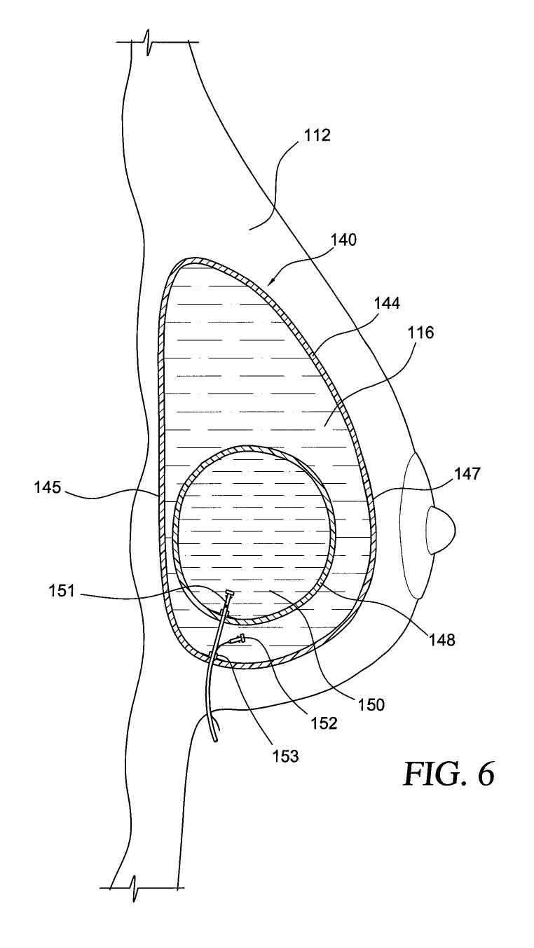

As shown in FIG. 2 an improved breast implant 20 in accordance with one embodiment of the invention includes a mesh pouch 22 of a light weight partially absorbable monofilament material and a non-absorbable silicone shell 23 that defines a hollow core 24 that is filled with silicone gel 25 or the like. The implant also includes an extension 26 of the mesh pouch 22 that is sutured to a patient's tissue. The implant includes a tube (not shown) leading to the pouch, but held within the surgeons hand. The remote port and tube are of conventional design and typically used to insert additional saline or silicone fluid or reduce the fluid filler from the inner prosthesis. The inner non-absorbable prosthesis or implant 23 is shown in FIGS. 4 and 5 wherein the inner prosthesis or inner implant 23 is encased in the mesh pouch 22. The mesh pouch is closed in an upward portion 25 to provide a fully enclosed structure.

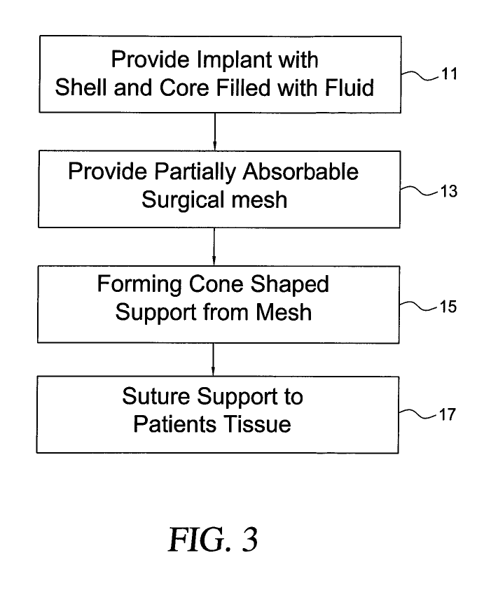

As illustrated in FIG. 3 a method in accordance with the present invention contemplates the following steps: The method includes the step 11 of providing an implant with an outer shell of medical grade silicone or the like having an inner core that is filled with a silicone gel, saline, etc. The method also includes the step 13 of providing a partially absorbable surgical mesh that is formed into a cone-shaped support in step 15 that is subsequently attached to a patient's tissue in step 17.

In FIG. 4, the pouch 22 is slightly spaced from the outer shell 23 in a manner that appears to minimize problems associated with capsular contraction.

It is also contemplated that rather than stitching the implant to the patient's tissue, the implant may be readily attached by a plurality of absorbable hooks 27 as shown in FIG. 5.

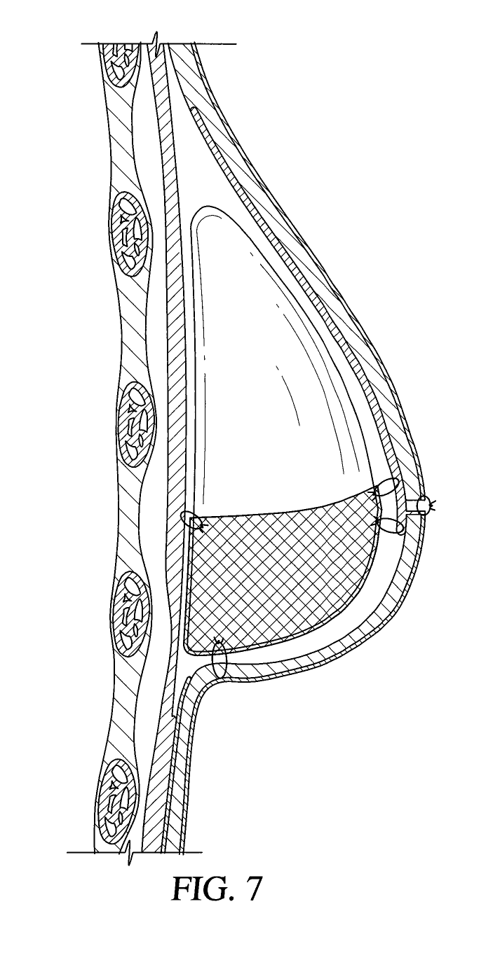

A preferred embodiment of the invention is illustrated in FIG. 7 wherein a self supporting breast implant comprises an adjustable implant having an upper and a lower portion and a generally cup or sling shaped mesh support surrounding and supporting said lower portion of said implant while leaving said upper portion thereof free of said mesh support and wherein said mesh support is adapted to hold the implant in position with respect to a muscle underlying an incision without piercing the implant. Further, the implant is susceptible to limited expansion in all directions and wherein the implant has a smooth surface, is expanded and subsequently reduced in volume and preferable is partially absorbable in the human body. For example, the mesh sling comprises a lightweight partially absorbable monofilament material that is made of silicone and filled with a mass of silicone, saline, or gel. In the most preferred embodiment of the invention the mesh support comprises about 25-75% polypropolene and about 75-25 wgt. % poliglecaprone monofilament. The non-absorbable portion may be even of a lower percentage.

The invention also contemplates a method for breast augmentation by implanting a self-supporting adjustable breast implant in a patient. The method includes the steps of providing an implant having an upper and a lower portion with a generally cup or sling shape support surrounding and supporting the lower portion of the implant by leaving the upper portion thereof free of the mesh support and wherein the implant is positioned with respect to a muscle underlying an incision without piercing the implant.

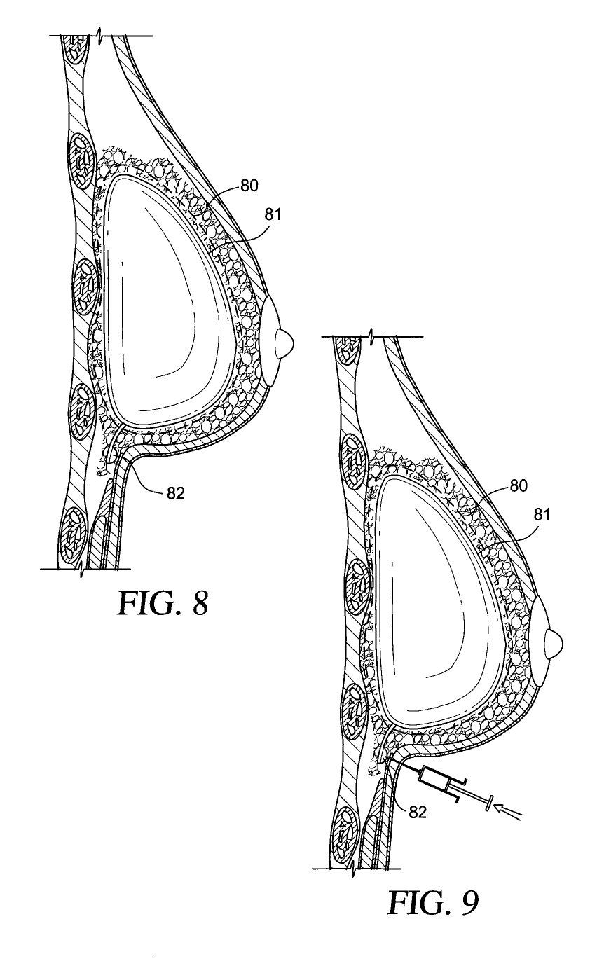

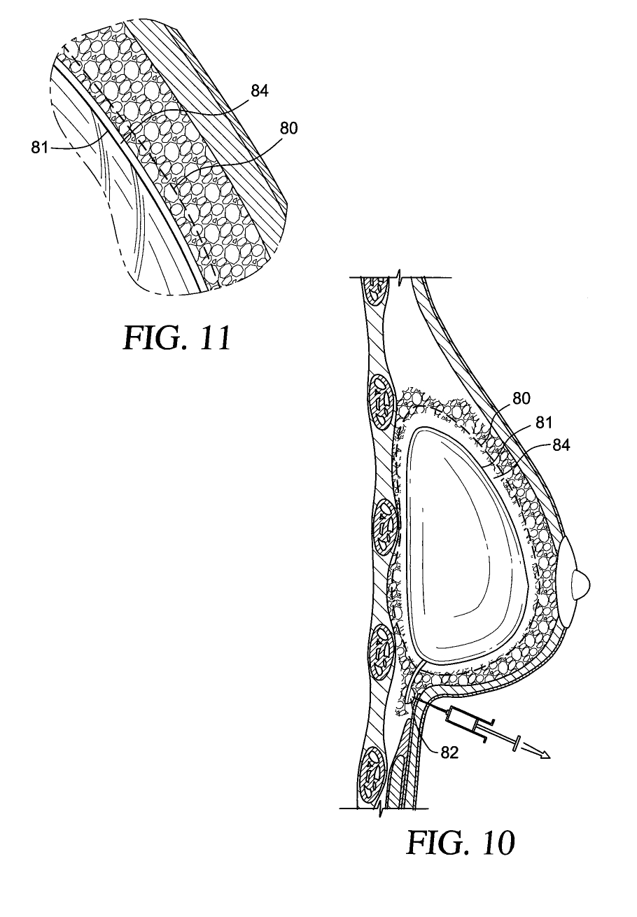

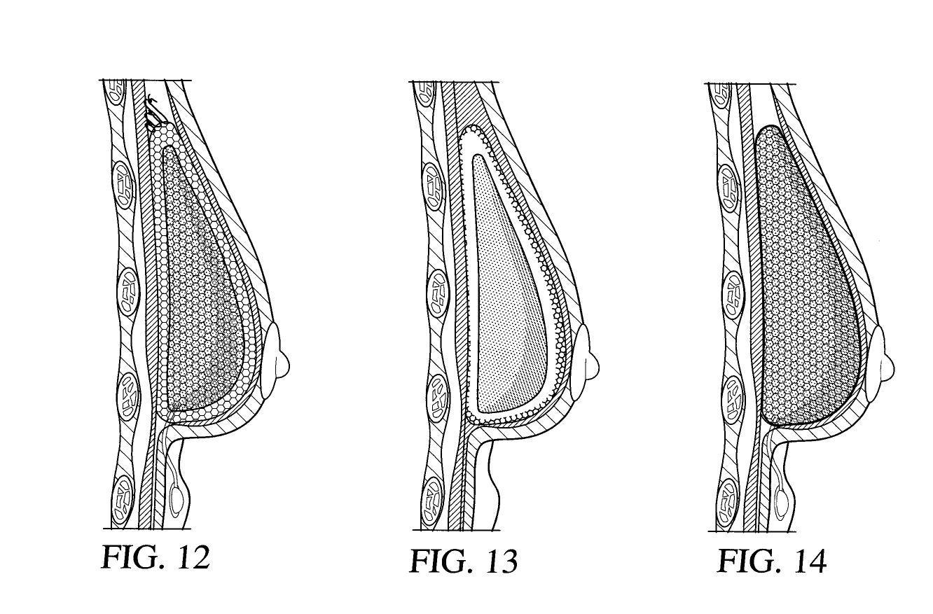

An implant as shown in FIG. 8 includes a mesh envelope 80 surrounding the shell of the implant 81 as implanted in a human breast. The implant or shell 81 is then expanded as shown in FIG. 9 by injecting saline or the like into the implant or shell 81 by means of an injection port 82. This injection increases the volume of the implant 81 and forces the mesh 80 into the surrounding tissue. After the mesh is adhered to the human tissue, the volume of the implant 81 is reduced by withdrawing fluid from or through the injection port 82. This leaves a space 84 between the implant 81 and a capsular tissue surface as shown in FIGS. 10 and 11.

The invention further contemplates coating the non-absorbable mesh or impregnating the absorbable mesh with an antibiotic.

While the invention has been described in connection with its preferred embodiments, it should be recognized that changes and modifications may be made therein without departing from the scope of the appended claims.

What is claimed is:

1. A self supporting breast implant comprising a generally cone shaped mesh support and a breast implant comprising a synthetic shell, a hollow core and a mass of silicone or saline fluid or gel disposed within said cone shaped support and wherein said cone shaped support includes attachment means for attaching said support to a patient's tissue without piercing the said implant.

2. A self supporting breast implant according to claim 1 in which said cone shaped support includes a relatively flat back surface attached to a base of said cone shaped support.

3. A self supporting breast implant according to claim 2 in which said breast implant has a smooth surface enclosed within said mesh support and said mesh is free of the implant in order to allow in-growth of the tissue into the mesh, without adherence to the implant.

4. A self supporting breast implant according to claim 3 in which there is a slight distance between said breast implant and said mesh support.

5. A self supporting breast implant according to claim 3 in which said implant is made of silicone.

6. A self supporting breast implant according to claim 4 in which said implant is a multi-lumen implant round or pear shaped and adjustable in order to expand mesh support to a larger size to that of the implant and then to be reduced in volume.

7. A self supporting breast implant according to claim 5 in which said mesh comprises a lightweight partially absorbable monofilament material or braided synthetic or biological combination material.

8. A self supporting breast implant according to claim 7 in which said mesh consists of about 10-90% polypropolene or other non-absorbable material and 10-90% poliglecaprone or other absorbable material impregnated with an antibiotic material.

9. A method for forming a support for a breast implant comprising the steps of:
providing a breast implant and a mass of surgical mesh;
forming a generally cone shaped mesh support; and
attaching said mesh support to a patient's tissue without penetrating the implant.

10. A method for forming and supporting a breast implant, said method consists of the following steps:
providing a silicone breast implant having a silicone elastomer shell including an outer layer of smooth silicone elastomer, a hollow core and a silicone or saline fluid or gel disparsed within said hollow core;
a mass of a light weight partially absorbable flexible monofilament materials;
forming a support member from said monofilament materials and impregnating said support member with an antiobiotic;
placing the breast implant within the cone shaped support member and closing said support member about said implant; and
attaching the support member to a patient's tissue without penetrating the implant.

11. A self supporting breast implant for breast augmentation consisting of:
a generally cone shaped mesh support made of about 75% polypropylene and 25% poliglecaprone monofilaments;
a silicone elastomer shell including a smooth outer surface layer of silicone elastomer, a hollow core and silicone or saline fluid or gel dispersed with said hollow core; and
wherein said cone shaped support includes attachment means wherein said attachment means are absorbable hooks fixed to a rear portion of said mesh pouch.

12. A self supporting breast implant comprising an implant having an upper and a lower portion and a generally cup or sling shaped support surrounding and supporting said lower portion of said implant while leaving said upper portion thereof free of said mesh support and wherein said mesh support and is adapted to hold said implant in position with respect to a muscle underlying an incision without piercing said implant and when said mesh support is impregnated with an antiobiotic.

13. A self supporting breast implant according to claim 12 wherein said breast support is susceptible to limited expansion in all directions.

14. A self supporting breast implant according to claim 13 in which said implant has a smooth surface and an expandable and/or reducible volume.

15. A self supporting breast implant according to claim 14 wherein said mesh support is partially absorbable in the human body.

16. A self supporting breast implant according to claim 15 wherein said mesh comprises a light weight partially absorbable monofilament material.

17. A self supporting breast implant according to claim 16 wherein said implant has a smooth surface, is made of silicon and filled with a mass of silicon, saline or gel.

18. A self supporting breast implant according to claim 17 in which said mesh support comprises about 75% wgt. polypropolene and about 25% wgt. poliglecaprone monofilament.

19. A self supporting breast implant consisting of a breast implant having an expandable and reducible smooth synthetic shell, a hollow core and a mass of silicon or saline fluid or gel disposed within said implant and wherein said implant has a generally round or pear shape with an upper and a lower portion and a generally cup of sling shaped mesh support surrounding and supporting said lower portion of said implant while leaving said upper portion thereof free of said mesh support and wherein said mesh support is partially absorbable in a human body and consists of synthetic, non-absorbable, absorbable monofilament, or braided in varying combinations, or combined with biological material and wherein said implant is a tissue expander or an adjustable implant.

20. A method for breast augmentation by implanting a self supporting breast implant in a patient said method including the steps of providing an implant having an upper and a lower portion with a generally cup or sling shape support surrounding and supporting said lower portion of said implant while leaving said upper portion thereof free of said mesh support and wherein said implant is positioned with respect to a muscle underlying an incision without piercing said implant, implanting said implant in a human body and expanding said implant by injecting a mass of fluid or gel into the implant allowing initial encapsulation and reducing the amount of fluid or gel to thereby reduce the size of the implant to thereby provide a soft pliable implant without surrounding tissue capsular contracture.
